(19) United States
(12) Patent Application Publication
(10) Pub. No.: US 2010/0166689 A1
(43) Pub. Date: Jul. 1, 2010

(54) Compositions and Methods for Treating Hyperpigmentation

(75) Inventor: Jacob M. Waugh, San Francisco, CA (US)

Correspondence Address: KING & SPALDING, 1185 AVENUE OF THE AMERICAS, NEW YORK, NY, 10036-4003, US

(73) Assignee: Revance Therapeutics, Inc., Newark, CA (US)

(21) Appl. No.: 12/647,739

(22) Filed: Dec. 28, 2009

Related U.S. Application Data
(60) Provisional application No. 61/142,094, filed on Dec. 31, 2008.

(51) Int. Cl. A61K 8/36; A61Q 19/02

(52) U.S. Cl. 424/62

(57) ABSTRACT

This invention provides compositions and methods for reducing hyperpigmentation. In preferred embodiments, the compositions are topical compositions that contain kojic acid and a carrier molecule for enhancing the transdermal penetration of kojic acid. This invention also provides kits for treating hyperpigmentation.

### RELATED PATENT APPLICATION

This application claims the benefit of priority under 35 U.S.C. §119 to U.S. Provisional Patent Application No. 61/142,094, filed Dec. 31, 2008, the contents of which are incorporated herein by reference in its entirety.

### FIELD OF THE INVENTION

The invention relates to treatment of hyperpigmentation and other undesirable pigmentation in skin. The invention provides compositions and methods for improved delivery of therapeutic agents for treating hyperpigmentation and other undesirable pigmentation in skin.

### BACKGROUND OF THE INVENTION

Melanin is a general term for a class of compounds that is found in animals, plants and protista. In humans, melanin is formed in melanosomes, which are cellular structures found in cells called melanocytes that are located in the lowest layer of the epidermis, the stratus basale, and basal cells. Melanin is transported via keratinocytes of the epidermis to corneocytes in the horny layer of skin, where it imparts a brownish pigment to the horny layer of skin. As the result of its presence in the horny layer of skin, melanin is responsible for pigmentation of human skin.

Melanin is capable of absorbing ultraviolet radiation, thereby playing an important role in protecting the human body, especially the skin, from the damaging and potentially carcinogenic effects of sunlight and other environmental sources of ultraviolet radiation. Upon exposure to ultraviolet radiation, the human body naturally increases the production of melanin in the exposed areas of skin as a defense mechanism. This increased production of melanin leads to a darkening of the exposed skin, a phenomenon which is commonly known as sun tanning. In some cultures, the darkened skin associated with sun tanning is considered to be desirable and aesthetically pleasing.

However, increased melanin production is not always viewed as desirable. For example, in some cultures, fair skin is considered more attractive than suntanned skin. Moreover, certain skin disorders can lead to uneven production of melanin in the skin, thereby causing the appearance of uneven skin pigmentation. For instance, hyperpigmentation disorders are characterized by localized darkening of skin color caused by locally high levels of melanin [e.g., see Voet D., Voet J. G., Pratt C W. Fundamentals of Biochemistry. New York: Von Hoffmann Press, 2001: 657]. Hyperpigmentation may be caused by either increased melanin production by existing melanocytes or proliferation of active melanocytes.

Hyperpigmentation and other conditions of uneven skin pigmentation are usually viewed as undesirable and unattractive. For instance, the occurrence of acne, rashes, scratch marks or injuries to the skin can result in post-inflammatory hyperpigmentation characterized by the presence of unwanted dark spots on the face or other parts of the body. Melasma, a condition associated with hormonal changes resulting from pregnancy, ingestion of birth control pills, or menopausal changes, is often concealed by depositing pigments superficially in the epidermis or deeper in the dermis. Lentigines, also known as liver spots, are dark discolorations due to sun damage which typically appear in older individuals. Ephelides, which are more commonly known as freckles, are small patches often seen in young people who have light-complexioned skin that has a tendency to burn when exposed to the sun [Cage K A and Feldman S T. Hyperpigmentation: A review of common treatment options. J. Drugs Dermatol. 2004; 3:668-678].

The skin discoloration associated with hyperpigmentation or sun tanning may be reduced by topically applying hydroquinone, a bleaching agent. Hydroquinone has been approved by the United States Food and Drug Administration (FDA) for gradually fading dark discolorations in the skin and is available in over-the-counter (OTC) skin bleaching formulations at concentrations up to 2% and in prescription formulations at concentrations of 3-4%. More recently, however, studies have been performed that call into question the safety of hydroquinone. These studies, currently under review by the FDA, have shown evidence of toxicity, carcinogenicity in animals, and occurrence of exogenous ochronosis in humans. It is possible that the FDA may ban the use of hydroquinone for treating hyperpigmentation.

In view of the potential safety hazards of using hydroquinone, it is desirable to develop compositions and methods for lightening skin color due to hyperpigmentation or sun tanning that do not involve hydroquinone. However, the skin-lightening agents identified thus far as possible alternatives to hydroquinone tend to have either low efficacy or undesirable side effects, such as, for example, toxicity or skin irritation. For example, while kojic acid has found some use as a skin-lightening agent, conventional topical kojic acid formulations for treating hyperpigmentation suffer from certain drawbacks. Because kojic acid does not penetrate human skin readily, conventional kojic acid formulations contain relatively high concentrations of kojic acid in order to provide enough transdermal flux in order to achieve a skin-lightening effect. At high concentrations, however, kojic acid is known to be an irritant with sensitization potential and to provoke contact dermatitis. Moreover, the high concentrations of kojic acid necessary to achieve a skin-lightening effect with conventional topical kojic acid-containing formulations poses potentially severe health risks, as some studies have indicated that high doses of kojic acid may be mutagenic and/or promote tumor formation. As a result of these drawbacks, some countries have placed a partial ban on the use of currently existing kojic acid formulations for reducing skin pigmentation associated with excess melanin.

Therefore, there is a need for new, safer formulations for treating hyperpigmentation.

### SUMMARY OF THE INVENTION

This invention provides compositions and methods for reducing the intensity of melanin-associated pigmentation of skin. The compositions and methods of the invention may be used to treat any condition associated with increased melanin production, including hyperpigmentation and sun tanning.

One embodiment of this invention provides a topical composition for treating hyperpigmentation. The composition comprises a skin-lightening agent and a positively charged carrier that is present in an amount sufficient to enhance transdermal transport of the skin-lightening agent. In preferred embodiments, the skin-lightening agent is kojic acid or a derivative of kojic acid.

Another embodiment of this invention provides a method for reducing the pigmentation of the skin. The method comprises identifying an area of skin to be treated and applying a composition to reduce the pigmentation of the skin the chosen area. The composition comprises a skin-lightening agent and a positively charged carrier molecule that is present in an amount to enhance transdermal transport of the skin-lightening agent. In preferred embodiments, the skin-lightening agent comprises kojic acid or a derivative of kojic acid.

Yet another aspect of the invention is to provide a kit for reducing the pigmentation of skin. The kit comprises a skin-lightening agent and a positively charged carrier that is present in an amount to enhance transdermal transport of the skin-lightening agent. The skin-lightening agent and the positively charged carrier may be stored separately as kit components and combined immediately prior to use or pre-mixed.

### DETAILED DESCRIPTION OF THE INVENTION

This invention provides a composition for reducing unwanted pigmentation in the skin, such as the pigmentation associated with hyperpigmentation or unwanted skin darkening, such as from sun tanning. In preferred embodiments, the compositions according to the invention comprise a skin-lightening agent and a delivery molecule capable of enhancing dermal penetration of the skin-lightening agent following topical application. The invention also provides a method for reducing the skin discolorations associated with hyperpigmentation by topically applying a skin-lightening agent and a delivery molecule capable of enhancing dermal penetration of the skin-lightening agent.

The skin-lightening agents contemplated by the invention are not particularly limited and include both synthetic and naturally occurring compounds that are capable of reducing discoloration associated with excess melanin. Non-limiting examples of skin-lightening agents contemplated by the invention include kojic acid, azelaic acid, ascorbic acid, tretinoin (Retinol), topical glucocorticoids, linoleic acid, niacinimide, 4-t-butyl catechol, tranexamic acid, and licorice extract. Combinations of skin-lightening agents are also contemplated by the invention.

In preferred embodiments, the skin-lightening agent is kojic acid or a derivative of kojic acid. Kojic acid (C₆H₆O₄; 5-hydroxy-2-(hydroxymethyl)-4-pyrone) has the following chemical structure

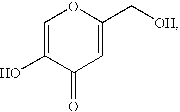

and may be obtained from a type of fungus known in Japan as koji (Asperigillus oryzae). Kojic acid blocks the formation of melanin by inhibiting the activity of tyrosinase, an enzyme that catalyzes in vivo chemical reactions associated with the formation of melanin. Kojic acid acts as a skin-lightening agent by preventing the synthesis of melanin. In certain preferred embodiments of the invention, the skin-lightening agent is a kojic acid derivative. As used herein, the term “kojic acid derivative” refers to kojic acid that has been subjected to one or more chemical or functional alterations, but which nonetheless possesses the ability to lighten skin discolorations caused by undesirably high levels of melanin. Kojic acid derivatives with the ability to lighten skin discolorations have been previously reported (e.g., see U.S. Pat. Nos. 5,486,624; 5,523,421; 5,824,327; and 5,968,487; the contents of which are incorporated by reference in their entirety). Non-limiting examples of kojic acid derivatives contemplated by the invention include 2-(2-hydroxybenzoyl)oxymethyl-5-hydroxy-4H-pyran-4-one, 2-(3-hydroxybenzoyl)oxymethyl-5-hydroxy-4H-pyran-4-one, 2-(4-hydroxybenzoyl)oxymethyl-5-hydroxy-4H-pyran-4-one, 2-(2,3-dihydroxybenzoyl)oxymethyl-5-hydroxy-4H-pyran-4-one, and 2-(3,4-dihydroxybenzoyl) oxymethyl-5-hydroxy-4H-pyran-4-one.

One aspect of this invention is the recognition kojic acid does not readily reach the necessary skin-associated structures to suppress melanin production. Without wishing to be limited by theory, it is believed that it is the difficulty in transporting kojic acid to the relevant skin-associated structures that has led to the high concentrations of kojic acid found in certain kojic acid formulations. Accordingly, preferred embodiments of this invention provide for positively charged carrier molecules that are capable of enhancing transdermal flux of kojic acid and/or directing kojic acid to the relevant skin-associated structures. The transport occurs without covalent modification of the skin-lightening agent.

By “positively charged” is meant that the carrier has a positive charge under at least some solution-phase conditions, more preferably under at least some physiologically compatible conditions More specifically, “positively charged” as used herein, means that the group in question contains functionalities that are charged under all pH conditions, for instance, a quaternary amine, or contains a functionality which can acquire positive charge under certain solution-phase conditions, such as pH changes in the case of primary amines. More preferably, “positively charged” as used herein refers to those groups that have the behavior of associating with anions over physiologically compatible conditions. Polymers with a multiplicity of positively-charged moieties need not be homopolymers, as will be apparent to one skilled in the art. Other examples of positively charged moieties are well known in the prior art and can be employed readily, as will be apparent to those skilled in the art.

Generally, the positively-charged carrier comprises a positively charged backbone, which is typically a chain of atoms, either with groups in the chain carrying a positive charge at physiological pH, or with groups carrying a positive charge attached to side chains extending from the backbone. Preferably, the positively charged backbone itself will not have a defined enzymatic or therapeutic biologic activity. The linear backbone is a hydrocarbon backbone which is, in some embodiments, interrupted by heteroatoms selected from nitrogen, oxygen, sulfur, silicon and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like) but can be a heteropolymer. In one group of embodiments, the positively charged backbone is a polypropyleneamine wherein a number of the amine nitrogen atoms are present as ammonium groups (tetra-substituted) carrying a positive charge. In another embodiment, the positively charged backbone is a nonpeptidyl polymer, which may be a hetero- or homo-polymer such as a polyalkyleneimine, for example a polyethyleneimine or polypropyleneimine, having a molecular weight of from about 100 to about 2,500,000 D, preferably from about 250 to about 1,800,000 D, and most preferably from about 1000 to about 1,400,000 D. In another group of embodiments, the backbone has attached a plurality of side-chain moieties that include positively charged groups (e.g., ammonium groups, pyridinium groups, phosphonium groups, sulfonium groups, guanidinium groups, or amidinium groups). The sidechain moieties in this group of embodiments can be placed at spacings along the backbone that are consistent in separations or variable. Additionally, the length of the sidechains can be similar or dissimilar. For example, in one group of embodiments, the sidechains can be linear or branched hydrocarbon chains having from one to twenty carbon atoms and terminating at the distal end (away from the backbone) in one of the above-noted positively charged groups. In all aspects of the present invention, the association between the carrier and the biologically active agent is by non-covalent interaction, non-limiting examples of which include ionic interactions, hydrogen bonding, van der Waals forces, or combinations thereof.

In one group of embodiments, the positively charged backbone is a polypeptide having multiple positively charged sidechain groups (e.g., lysine, arginine, ornithine, homoarginine, and the like). Preferably, the polypeptide has a molecular weight of from about 100 to about 1,500,000 D, more preferably from about 250 to about 1,200,000 D, most preferably from about 1000 to about 1,000,000 D. One of skill in the art will appreciate that when amino acids are used in this portion of the invention, the sidechains can have either the D- or L-form (R or S configuration) at the center of attachment. In certain preferred embodiments, the polypeptide has a molecular weight from about 500 to about 5000 D, more preferably from 1000 to about 4000 D, more preferably from 2000 to about 3000 D. In other embodiments, the polypeptide has a molecular weight of at least about 10,000.

In another embodiment, the backbone portion is a polylysine and efficiency groups, as discussed herein, are attached to the polylysine. The polylysine may have a molecular weight of from about 100 to about 1,500,000 D, preferably from about 250 to about 1,200,000 D, and most preferably from about 1000 to about 3000 D. It also can be any of the commercially available (Sigma Chemical Company, St. Louis, Mo., USA) polylysines such as, for example, polylysine having MW>70,000 D, polylysine having MW of 70,000 to 150,000 D, polylysine having MW 150,000 to 300,000 D and polylysine having MW>300,000 D. The selection of an appropriate polylysine will depend on the remaining components of the composition and will be sufficient to provide an overall net positive charge to the composition and provide a length that is preferably from one to four times the combined length of the negatively charged components.

Alternatively, the backbone can be an analog of a polypeptide such as a peptoid. See, for example, Kessler, Angew. Chem. Int. Ed. Engl. 32:543 (1993); Zuckermann et al. Chemtracts—Macromol. Chem. 4:80 (1992); and Simon et al. Proc. Nat'l. Acad. Sci. USA 89:9367 (1992)). Briefly, a peptoid is a polyglycine in which the sidechain is attached to the backbone nitrogen atoms rather than the alpha-carbon atoms. As above, a portion of the sidechains will typically terminate in a positively charged group to provide a positively charged backbone component. Synthesis of peptoids is described in, for example, U.S. Pat. No. 5,877,278, which is hereby incorporated by reference in its entirety. As the term is used herein, positively charged backbones that have a peptoid backbone construction are considered “non-peptide” as they are not composed of amino acids having naturally occurring sidechains at the α-carbon locations.

A variety of other backbones can be used employing, for example, steric or electronic mimics of polypeptides wherein the amide linkages of the peptide are replaced with surrogates such as ester linkages, thioamides (—CSNH—), reversed thioamide (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene (—CR═CH—), fluoroalkene (—CF═CH—), dimethylene (—CH₂CH₂—), thioether (—CH₂S—), hydroxyethylene (—CH(OH)CH₂—), methyleneoxy (—CH₂O—), tetrazole (CN₄), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from .alpha.-amino acids.

In each of the backbones provided above, sidechain groups can be appended that carry a positively charged group. For example, the sulfonamide-linked backbones (—SO₂NH— and —NHSO₂—) can have sidechain groups attached to the nitrogen atoms. Similarly, the hydroxyethylene (—CH(OH)CH₂—) linkage can bear a sidechain group attached to the hydroxy substituent. One of skill in the art can readily adapt the other linkage chemistries to provide positively charged sidechain groups using standard synthetic methods.

In one embodiment of the invention, only a positively charged carrier that has positively charged efficiency groups is necessary for transdermal delivery of the skin-lightening agent. In certain embodiments, the positively charged backbone is a polypeptide (e.g., lysine, arginine, ornithine, homoarginine, and the like) having multiple positively charged side-chain groups, as described above. In another embodiment, the positively charged carrier comprises a nonpeptidyl polymer such as a polyalkyleneimine having multiple positively charged side-chain groups having a molecular weight in the range of about 100 to 1,500,000 D. Such polyalkyleneimines include polyethylene- and polypropyleneimines.

In preferred embodiments, the positively charged carrier comprises a positively charged backbone with a plurality of attached efficiency groups. As used herein, an “efficiency group” is any agent that has the effect of promoting the translocation of the positively charged backbone through a tissue or cell membrane. Non-limiting examples of efficiency groups include -(gly)ₙ₁-(arg)ₙ₂, HIV-TAT or fragments thereof, or the protein transduction domain of Antennapedia, or a fragment thereof, in which the subscript n1 is an integer of from 0 to 20, more preferably 0 to 8, still more preferably 2 to 5, and the subscript n2 is independently an odd integer of from about 5 to about 25, more preferably about 7 to about 17, most preferably about 7 to about 13. Still further preferred are those embodiments in which the HIV-TAT fragment has the formula (gly)ₚ-RGRDDRRQRRR-(gly)_{q}, (gly)ₚ-YGRKKRRQRRR-(gly)_{q} or (gly)ₚ-RKKRRQRRR-(gly)_{q} wherein the subscripts p and q are each independently an integer of from 0 to 20 and the fragment is attached to the backbone via either the C-terminus or the N-terminus of the fragment. Preferred HIV-TAT fragments are those in which the subscripts p and q are each independently integers of from 0 to 8, more preferably 2 to 5. In another preferred embodiment the efficiency group is the Antennapedia (Antp) protein transduction domain (PTD), or a fragment thereof that retains activity. These are known in the art, for instance, from Console et al., J. Biol. Chem. 278:35109 (2003) and a non-limiting example of a Antp PTD contemplated by this invention is SGRQIKIWFQNRRMKWKKC. Preferably the positively charged carrier includes efficiency groups in an amount of at least about 0.05%, as a percentage of the total carrier weight, preferably from about 0.05 to about 45 weight %, and most preferably from about 0.1 to about 30 weight %. For positively charged efficiency groups having the formula -(gly)ₙ₁-(arg)ₙ₂, the most preferred amount is from about 0.1 to about 25%. Preferred positively charged efficiency groups include, for example, -gly-gly-gly-arg-arg-arg-arg-arg-arg-arg (-Gly₃Arg₇), HIV-TAT or fragments of it, and Antennapedia PTD or fragments thereof.

In other embodiments of this invention, the positively charged carrier is a relatively short polylysine or polyethyleneimine (PEI) backbone (which may be linear or branched) and which has positively charged efficiency groups. A non-limiting example of such a carrier is the amino acid sequence RKKRRQRRRG-(K)₁₅-GRKKRRQRRR. In preferred embodiments, such carriers are useful for minimizing uncontrolled aggregation of the backbones and skin-lightening agent in a therapeutic composition, which causes the transport efficiency to decrease dramatically. In some embodiments, when the carrier is a relatively short linear polylysine or PEI backbone, the backbone will have a molecular weight of less than 75,000 D, more preferably less than 30,000 D, and most preferably, less than 25,000 D. For example, in certain embodiments, the carrier is a relatively short branched polylysine or PEI backbone with a molecular weight less than 60,000, more preferably less than 55,000 D, and most preferably less than 50,000 D.

Compositions of this invention are preferably in the form of products to be applied to the skin of subjects or patients, i.e. humans or other mammals in need of the particular treatment. The term “in need” is meant to include both pharmaceutical or health-related needs, as well as cosmetic and subjective needs, for example, altering or improving the appearance of facial tissue. In general the compositions are prepared by mixing the skin-lightening agent with the positively charged carrier, and optionally with one or more additional pharmaceutically acceptable carriers or excipients. In their simplest form they may contain a simple aqueous pharmaceutically acceptable carrier or diluent, such as buffered saline (e.g., phosphate buffered saline). However, the compositions may contain other ingredients typical in topical pharmaceutical or cosmeceutical compositions, including a dermatologically or pharmaceutically acceptable carrier, vehicle or medium, (i.e. a carrier, vehicle or medium that is compatible with the tissues to which they will be applied.) The term “dermatologically or pharmaceutically acceptable,” as used herein, means that the compositions or components thereof so described are suitable for use in contact with these tissues or for use in patients in general without undue toxicity, incompatibility, instability, allergic response, and the like. As appropriate, compositions of the invention may comprise any ingredient conventionally used in the fields under consideration, and particularly in cosmetics and dermatology. The compositions also may include a quantity of a small anion, preferably a polyvalent anion, for example, phosphate, aspartate, or citrate.

In terms of their form, compositions of this invention may include solutions, emulsions (including microemulsions), suspensions, creams, lotions, gels, powders, or other typical solid or liquid compositions used for application to skin and other tissues where the compositions may be used. Such compositions may contain, in addition to the skin-lightening agent and carrier, other ingredients typically used in such products, such as antimicrobials, moisturizers and hydration agents, penetration agents, preservatives, emulsifiers, natural or synthetic oils, solvents, surfactants, detergents, emollients, antioxidants, fragrances, fillers, thickeners, waxes, odor absorbers, dyestuffs, coloring agents, powders, and optionally including anesthetics, anti-itch additives, botanical extracts, conditioning agents, lightening agents, glitter, humectants, mica, minerals, polyphenols, silicones or derivatives thereof, sunblocks, vitamins, and phytomedicinals.

In particularly preferred embodiments, the compositions include gelling agents and/or viscosity-modifying agents. These agents are generally added to increase the viscosity of the composition, so as to make the application of the composition easier and more accurate. Additionally, these agents help to prevent the aqueous skin-lightening agent/carrier solution from drying out, which tends to cause a decrease in the activity of the skin-lightening agent. Particularly preferred agents are those that are uncharged and do not interfere with the skin-lightening agent activity or the efficiency of the toxin-carrier complexes in crossing skin. The gelling agents may be certain cellulose-based gelling agents, such as hydroxypropylcellulose (HPC) for example. In some embodiments, the skin-lightening agent/carrier complex is formulated in a composition having 2-4% HPC. Alternatively, the viscosity of a solution containing a skin-lightening agent/carrier complex may be altered by adding polyethylene glycol (PEG). In other embodiments, the skin-lightening agent/carrier solution is combined with pre-mixed viscous agents, such as Cetaphil® moisturizer.

This invention also contemplates kits comprising one or more skin-lightening agents and a positively charged carrier according to the invention. The one or more skin-lightening agents and positively charged carrier may be pre-mixed or may exist in the kit as separate components that are mixed prior to administration. The kit may include devices for delivering one or more skin-lightening agents and a positively charged carrier. Non-limiting examples of such a device include a skin patch and a custom applicator.

It is understood that the following examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

### In Vitro Testing—Tyrosinase Inhibition Assay

This example reports a comparative study in which the transdermal fluxes of kojic acid formulations (with and without an exemplary positively charged carrier molecule according to the invention) through porcine skin are measured. As discussed in detail below, the results show that the transdermal flux of kojic acid with the exemplary positively charged molecule of the invention is over a factor of two higher than the transdermal flux observed for a kojic acid formulation that is identical, with the exception that is does not contain the positively charged carrier molecule.

The assays reported in this example take advantage of the fact that the ability of the enzyme tyrosinase to oxidize phenols, such as tyrosine, is inhibited by the presence of kojic acid. By monitoring the extent to which the tyrosinase activity is reduced by kojic acid that has penetrated porcine skin, one can obtain a measure of the transdermal flux of the kojic acid corresponding to various kojic acid formulations. A high level of tyrosinase inhibition in these studies indicates a high level of transdermal flux of kojic acid through the porcine skin. The inhibition reaction was monitored using optical photometry, because the enzymatic reaction between tyrosine and tyrosinase is accompanied by a color change that can be monitored by measuring the optical density at 475 nm, which is proportional to the concentration of tyrosinase.

For the studies reported in this example, all reagents were obtained from Sigma-Aldrich, St. Louis. MO. A 1.8 mM L-tyrosine solution and 1.25 U/4 mushroom tyrosinase enzyme solution were prepared using 67 mM potassium phosphate buffer at a pH of 6.8. A polyaspartate flux buffer (concentration: 77 ng/ml) was prepared in PBS with 1% BSA. A positively charged carrier molecule having the formula RKKRRQRRRG-(K)₁₅-GRKKRRQRRR (hereafter “RTP004”) was made with 0.9% NaCl at 10 μg/μL concentration. RTP004 was synthesized using tBoc and/or Fmoc solid phase chemistry. A 0.625 mM kojic acid solution (molecular weight 142 g/mol) was prepared in both potassium phosphate buffer and polyaspartate flux buffer. The IC₅₀ value for kojic acid was calculated as 30 μM (4.26 μg/mL). Thus, a 2000-fold more concentrated kojic acid solution was used (i.e., about 4260 μg of kojic acid in 200 μl of buffer) in flux experiments to account for the dilution with buffer that occurs during collection of the kojic acid that passes through the porcine skin, as discussed below.

### Validation of the Kinetic Assay in Flux Buffer

Prior to using a kinetic assay to measure the transdermal flux of the kojic acid formulations through porcine skin, the kinetic assay was validated to confirm the sensitivity of the assay for detecting the inhibition of tyrosinase by kojic acid. The validation of the assay involved measuring the extent of inhibition of tyrosinase activity and dose dependent inhibition of tyrosinase activity by kojic acid at various time points and at successive serial dilutions. More specifically, 140 μL of 0.625 mM kojic acid, 35 μL of 1.8 mM of L-tyrosine, 25 μL of 1.25 U/μL mushroom tyrosinase enzyme were loaded into a 96-well plate, and incubated at 37° C. The kinetic assay was performed at 1, 2, 3, and 4 hour time points for 30 minutes, with optical density (OD) readings at 475 nm and results collected at every 1 minute intervals (SpectraMax M5, Molecular Devices, Sunnyvale, Calif.). Some of the wells of the 96-well plates were control wells into which flux buffer, without any tyrosinase or L-tyrosine, was added. To calibrate the amount of kojic acid present in the various reaction wells, a measured amount of kojic acid was spiked in flux buffer flow from control wells at every hour. These values were used to provide a positive control in order to estimate the amount of kojic acid in the reaction wells that contained a mixture of tyrosinase, tyrosine, and kojic acid.

### Franz Chamber Assay for Transdermal Flux

The transdermal flux of the kojic acid was measured for the following three formulations: (1) a formulation containing 4 mg of kojic acid in 200 μL of flux buffer with no positively charged carrier molecule; (2) a formulation containing 4 mg of kojic acid with the 12 micrograms of RTP004 in 200 μL of flux buffer; and (3) a control formulation containing in 200 μL of flux buffer, but no kojic acid or RTP004.

The transdermal flux of kojic acid associated with the three formulations was measured using a Franz chamber (PermeGear, Bethlehem, Pa.; Isco Retriever IV, Lincoln, Nebr.). Briefly, a Franz chamber is a device that permits the measurement of flux of a compound through a membrane (here, porcine skin). Each of the formulations to be studied was placed on one side of the porcine skin and allowed to diffuse through to the other side for four hours. The solutions that pass through the porcine skin (i.e., the flow-through solutions) enter a stream of continuously circulating 0.9% NaCl buffer, which is ultimately collected and analyzed in a kinetic assay.

More specifically, in-line cells were assembled to the Franz Chamber and a circulator reservoir was filled with 0.9% NaCl. Porcine skin (0.45 mm thickness) was loaded into the in-line cells, and 200 μL of the three formulations was added to each cell. The Franz Chamber ran at 8 μL/minute (min) for 4 hours, with a shuttle change once per hour (total of 480 μt per sample) for a total of 5 samples per group. Three different flux samples were tested in a total volume of 200 μL/cell (N=5 per sample). Note that samples were mixed and incubated at room temperature for 5 minutes before they were loaded on to each respective Franz cell.

The flow-through solutions obtained for each of the three formulations were collected and subjected to a kinetic assay. In the kinetic assay, each well in a 96-well plate was loaded with 35 μL of 1.8 mM of L-tyrosine, 25 μL of 1.25 U/μL mushroom tyrosinase enzyme, and 140 μL of sample. The sample solutions were either the flow-through solutions, as collected, or solutions obtained by subjecting the flow-through solutions to successive serial two-fold dilutions as indicated in Table 3 (n=5 for each concentration). Kojic acid plus peptide delivery was measured by the percentage of applied load appearing in the flow-through solution. For the positive control, no kojic acid was added.

The results indicate that the formulation containing both kojic acid and RTP004 exhibit greater transmembrane flux of kojic acid than the formulation that contained kojic acid, but no RTP004. More specifically, the percentage of the kojic acid that passed through the porcine skin, relative to the applied load of kojic acid, was determined to be 12.28% for the formulation containing kojic acid and RTP004, but was only 5.62% for the formulation containing kojic acid alone. As expected, no kojic acid flux was observed for the control formulation.

Thus, this example indicates that the transdermal flux of kojic acid may be enhanced by using positively charged carrier molecules according to the invention. This result indicates that topical compositions according to the invention may permit equivalent transdermal flux of a skin-lightening agent, such as kojic acid, even with a lower concentration of the skin-lightening agent in the topical composition. The compositions of the invention therefore may help to mitigate the harmful effects caused by topical high concentration of kojic acid.

What is claimed is:

1. A topical composition comprising
a skin lightening agent, and
a positively charged carrier molecule comprising a positively charged backbone and a plurality of efficiency groups attached thereto.

2. The topical composition according to claim 1, wherein the skin-lightening agent is selected from the group consisting of kojic acid, derivative of kojic acid, azelaic acid, ascorbic acid, tretinoin (Retinol), topical glucocorticoids, linoleic acid, niacinimide, 4-t-butyl catechol, tranexamic acid, and licorice extract.

3. The topical composition according to claim 2, wherein the skin-lightening agent is kojic acid.

4. The topical composition according to claim 1, wherein the positively charged backbone is a polyamino acid or a polyalkyleneimine.

5. The topical composition according to claim 4, wherein the polyamino acid is selected from the group consisting of polylysine, polyarginine, polyhistidine, and polyornithine.

6. The topical composition according to claim 1, wherein efficiency groups are amino acid sequences selected from the group consisting of -(gly)ₙ₁-(arg)ₙ₂, HIV-TAT or fragments thereof, or Antennapedia PTD or a fragment thereof, (gly)ₚ-RGRDDRRQRRR-(gly)_{q}, (gly)ₚ-YGRKKRRQRRR-(gly)_{q}, and (gly)ₚ-RKKRRQRRR-(gly)_{q},
wherein the subscript n1 is an integer of from 0 to 20 and the subscript n2 is independently an odd integer of from about 5 to about 25; and
wherein the subscripts p and q are each independently an integer of from 0 to 20.

7. A method of reducing pigmentation in skin, the method comprising
identifying a region of skin in need of treatment;
applying a topical composition to the region of skin in need of treatment, the topical composition comprising a skin-lightening agent and a positively charged carrier molecule, the positively charged carrier molecule comprising a positively charged backbone and a efficiency groups attached thereto.

8. The method according to claim 7, wherein the skin-lightening agent is selected from the group consisting of kojic acid, derivative of kojic acid, azelaic acid, ascorbic acid, tretinoin (Retinol), topical glucocorticoids, linoleic acid, niacinimide, 4-t-butyl catechol, tranexamic acid, and licorice extract.

9. The method according to claim 8, wherein the skin-lightening agent is kojic acid.

10. The method according to claim 7, wherein the positively charged backbone is a polyamino acid or a polyalkyleneimine.

11. The method according to claim 10, wherein the polyamino acid is selected from the group consisting of polylysine, polyarginine, polyhistidine, and polyornithine.

12. The method according to claim 7, wherein efficiency groups are amino acid sequences selected from the group consisting of -(gly)ₙ₁-(arg)ₙ₂, HIV-TAT or fragments thereof, or Antennapedia PTD or a fragment thereof, (gly)ₚ-RGRDDRRQRRR-(gly)_{q}, (gly)ₚ-YGRKKRRQRRR-(gly)_{q}, and (gly)ₚ-RKKRRQRRR-(gly)_{q},
wherein the subscript n1 is an integer of from 0 to 20 and the subscript n2 is independently an odd integer of from about 5 to about 25; and
wherein the subscripts p and q are each independently an integer of from 0 to 20.
